(19) United States
(12) Patent Application Publication
(10) Pub. No.: US 2005/0042281 A1
(43) Pub. Date: Feb. 24, 2005

(54) Compositions for delivering therapeutic agents across the oral mucosa

(76) Inventors: Nikhilesh N. Singh, Mill Valley, CA (US); Natasha N. Singh, Mill Valley, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/646,990

(22) Filed: Aug. 21, 2003

(51) Int. Cl. A61K 31/485; A61K 9/20

(52) U.S. Cl. 424/464; 514/282; 514/317

(57) ABSTRACT

Described herein are compositions for delivering a therapeutic agent across the oral mucosa. In general, the compositions comprise at least one therapeutic agent, a carrier, and a buffer system. The therapeutic agents may be acidic, basic, or amphoteric and are initially, at least in part, present in an ionized form. The buffer system comprises at least two different buffering agents and is capable of changing the pH of the saliva from an arbitrary initial pH to a predetermined final pH, independent of the arbitrary initial pH, and of sustaining the final pH for a predetermined period of time. In addition, the buffer systems described herein favor the substantially complete conversion of the ionized form of the therapeutic agent to the un-ionized form so that practically all of the therapeutic agent will be delivered rapidly across the mucous membranes of the oral cavity. Chewing gums, lozenges, and quick-dissolving tablets are exemplified.

### BACKGROUND

While there are various types of dosage forms, solid dosage forms for oral administration are perhaps among the most preferred by patients, and among the most prevalently used. These dosage forms are typically medicaments formulated as tablets, capsules, or liquids, which are swallowed. Oral administration, however, has several disadvantages, such as drug losses during hepatic first pass metabolism, during enzymatic degradation within the GI tract, and during absorption. These drug losses not only increase the variability in drug response, but also often require that the medicament be given in greater initial doses. In addition, because the drug has to pass through the gastrointestinal system in order to enter the blood stream, the time to reach a therapeutic effect may be quite long, typically around forty-five minutes or longer.

Accordingly, other routes of drug administration have been investigated, including those involving transport across the mucous membranes. Of the various mucous membranes (e.g., oral, rectal, vaginal, ocular, nasal, etc.), drug delivery via the mucous membranes in the oral cavity seem to be the most easily tolerated by patients. In addition to avoiding the problems with traditional oral administration, drug delivery via the mucous membranes of the oral cavity has certain other advantages, due to the properties of the oral mucosa itself. For example, the mucous membranes of the oral cavity are highly vascularized and well supplied with lymphatic drainage sites.

In general, the mucous membranes of the oral cavity can be divided into five main regions: the floor of the mouth (sublingual), the cheeks (buccal), the gums (gingival), the roof of the mouth (palatal), and the lining of the lips. These regions differ from each other with respect to their anatomy, drug permeability, and physiological response to drugs. For example, in terms of permeability, sublingual is more permeable than buccal, which is more permeable than palatal. This permeability is generally based on the relative thickness and degree of keratinization of these membranes, with the sublingual mucosa being relatively thin and non-keratinized, the buccal mucosa being thicker and non-keratinized, and the palatal mucosa being intermediate in thickness, but keratinized.

In addition to the differences in permeability of the various mucous membranes, the extent of drug delivery is also affected by the properties of the drug to be delivered. The ability of a molecule to pass through any mucous membrane is dependent upon its size, its lipid solubility, and the extent to which it is ionized, among other factors.

The extent to which a drug is ionized has further been investigated with respect to drug delivery. Ionization is dependant on the dissociation constant, or pKa of the molecule, and the pH of the molecule's surrounding environment. In its un-ionized form, a drug is sufficiently lipophilic to traverse a membrane via passive diffusion. In fact, according to the pH partition hypothesis, only un-ionized, non-polar drugs will penetrate a lipid membrane.

At equilibrium, the concentrations of the un-ionized form of the drug are equal on both sides of the membrane. Therefore, as the percentage of un-ionized form of a drug is increased, transmucosal absorption of the drug is correspondingly increased. Maximum absorption across the membrane is thought to occur when a drug is 100% in its un-ionized form. Similarly, absorption across the membrane decreases as the extent of ionization increases. Therefore, one may influence the extent of drug absorption across the mucous membranes of the oral cavity by altering the pH of saliva environment.

Some of the known transmucosal dosage forms include the use of a single buffering agent in order to change the pH of the saliva. However, these single buffering agents typically react with an acid or a base to create a final pH that is dependent upon the initial pH of the saliva of the user. A buffering agent used to attain a final pH that is dependent upon the initial pH of the user results in great variability. The extent of ionization, and hence the extent of absorption across the mucous membranes cannot be predicted with any sort of accuracy. This may pose significant problems when trying to calculate precise dosages, and in trying to prove consistency in drug loading to the regulatory authorities. In addition, a single buffering agent is typically not capable of sustaining a given pH over a period of time. While other investigators have disclosed the use of more than one buffering agent, these aforementioned problems are not easily cured by the nonchalant addition of an extra buffering agent. That is, a buffering system capable of achieving and sustaining a final pH, independent of the initial pH in order to increase transmucosal absorption, has not heretofore been demonstrated.

Similarly, a buffer system that facilitates substantially complete conversion of the ionized to the un-ionized form in a short period of time so as to cause rapid delivery of practically an entire drug dose across the oral mucosa has not heretofore been demonstrated. Previous dosage forms resulted in great variability in drug delivery, due to the variability in the rates in which a drug was released from its carrier. That is, the rates of drug release in previously described chewing gums or lozenges are largely dependent upon the rate of chewing or sucking of the user. The variability in these rates from user to user further exacerbates the ability to predict the final amount of drug that will enter systemic circulation. In addition, the rate of drug release from chewing gums is further dependent upon the ability of the drug to be released from the gum base. Often times, the gum base strongly adheres to the drug, making the at least portions of the drug unavailable for absorption.

Accordingly, compositions for delivering therapeutic agents across the oral mucosa having buffer systems that facilitate absorption of the agents would be desirable. Similarly, compositions for delivering therapeutic agents across the oral mucosa having a buffer system that produces a final pH, independent of the initial pH, and sustains that final pH for a given period of time would be desirable. In addition, compositions capable of rapidly facilitating substantially complete conversion of a therapeutic agent from its ionized to its un-ionized form would also be desirable. In addition, compositions that may be easily modified for use with a wide variety of therapeutic agents, and for a wide variety of dosage forms would be desirable.

### SUMMARY

Described herein are compositions for delivering at least one therapeutic agent across the oral mucosa. In general, the compositions comprise at least one therapeutic agent, a carrier, and a buffer system. In some variations, the compositions further comprise a penetration enhancer.

The at least one therapeutic agent is at least partly in an ionized form, and the ionized form is capable of being converted into an un-ionized form. The buffer system comprises at least two different buffering agents and is capable of changing the pH of saliva from an arbitrary initial pH to a predetermined final pH, independent of the arbitrary initial pH, and of sustaining the predetermined final pH for a period of time.

In some of the variations described herein, the at least one therapeutic agent is basic. In other variations, the at least one therapeutic agent is acidic or amphoteric. Examples of suitable therapeutic agents (described as un-ionized) for use in the compositions described herein, include alfentanil, allylprodine, alphaprodine, anileridine, benzylmorphine, bezitramide, clonitazene, codeine, dextromoramide, diampromide, dihydrocodeine, dimenoxadol, dimepheptanol, dimethylthiambutene, dioxaphetyl butyrate, dipipanone, ethoheptazine, ethylmethylthiambutene, ethylmorphine, etonitazene, fentanyl, heroin, hydrocodone, isomethadone, levophenacylmorphan, lofentanil, meperidine, methadone, morphine, narceine, nicomorphine, norlevorphanol, normethadone, norpipanone, opium, oxycodone, papaveretum, phenadoxone, phenoperidine, piminodine, piritramide, propheptazine, promedol, properidine, propiram, propoxyphene, sufentanil, tramadol, tilidine, montelukast, buprenorphine, butorphanol, cyclazocine, desomorphine, dezocine, dihydromorphine, eptazocine, hydromorphone, hydroxypethidine, ketobemidone, levallorphan, levorphanol, meptazinol, metazocine, metopon, morphine, nalbuphine, nalorphine, naloxone, naltrexone, normorphine, oxymorphone, pentazocine, phenomorphan, phenazocine, sumatriptan, zolmitriptan, naratriptan, rizatriptan, eletriptan, almotriptan, frovatriptan, analogs, and mixtures thereof.

In some variations employed with basic drugs, the buffer system changes the arbitrary initial pH to a predetermined final pH that is within a range of from about 7.1 to about 11.5. In other variations employed with acidic drugs, the range is from about 9 to about 11. In still other variations, the range is from about 2 to about 6.9. In yet other variations, the final pH is within a range from about 2 to about 4.

The carrier of the compositions described herein may provide for a variety of different dosage forms. For example, the carrier may provide for a chewing gum, lozenge, or quick-dissolving tablet dosage form. In some variations, the carrier is a gum base. The gum base may comprise at least one hydrophobic polymer and at least one hydrophilic polymer, which may be independently selected from the group consisting of a water-insoluble polymer, a natural polymer, a synthetic polymer, and mixtures thereof. In some variations, the hydrophobic polymer comprises a mixture of butyl rubber and polyisobutylene.

The buffer system favors substantially complete conversion of the ionized form to the un-ionized form. As used herein, the phrase substantially complete conversion means greater than 50% conversion of the ionized form into the un-ionized form. For example, the buffer system may favor at least 80%, at least 95%, or at least 99% conversion of the ionized form to the un-ionized form. In some variations, the conversion occurs in 10 minutes or less. The buffering agents of the buffer system may be independently selected from the group consisting of sodium carbonate, sodium bicarbonate, potassium carbonate, potassium bicarbonate, potassium citrate, potassium phosphate monobasic, magnesium oxide, magnesium carbonate, magnesium bicarbonate, alkaline starch, ascorbic acid, and mixtures thereof. In some variations, one buffering agent is sodium bicarbonate and one buffering agent is sodium carbonate. In other variations, one buffering agent is potassium bicarbonate and one buffering agent is potassium carbonate. The buffering agents may be combined in a ratio so as to produce the predetermined final pH, for example a 2 to 1 ratio by weight, a 3 to 1 ratio by weight, a 5 to 1 ratio by weight, a 10 to 1 ratio by weight, or 1 to 1 ratio by weight. In some variations, the period of time for sustaining the predetermined final pH is at least 5 minutes. In other variations, the period of time for sustaining the predetermined final pH is at least 10 minutes, or at least 20 minutes.

Chewing gum composition are also described herein. In general, the chewing gum compositions comprise at least one therapeutic agent, a gum base, a protecting agent, and a buffer system. The at least one therapeutic agent is at least partly in an ionized form, and the ionized form is capable of being converted into an un-ionized form. The protecting agent coats at least a portion of the at least one therapeutic agent and reduces adhesion between the therapeutic agent and the gum base. In some variations, the protecting agent comprises magnesium stearate.

The buffer system and the therapeutic agent may be of the same general nature as described above. In some variations, the chewing gum composition further comprises a binder, a filler, a flavoring agent, a scenting agent, a coloring agent, a preservative, a softening agent, a penetration enhancer, an elastomeric solvent, or mixtures thereof.

### BRIEF DESCRIPTION OF THE DRAWING

FIG. 1 is a graph showing plasma concentration comparisons over time between a transmucosally delivered sumatriptan solution at pH 10, and an orally administered sumatriptan tablet.

### DETAILED DESCRIPTION

Described herein are compositions for delivering therapeutic agents across the oral mucosa. In general, the compositions comprise at least one therapeutic agent, a carrier, and a buffer system. As described in more detail below, a variety of different therapeutic agents may be selected for delivery across the mucous membranes of the oral cavity. Similarly, the carrier may be selected so as to provide a variety of different dosage forms. For example, the compositions may be formulated as a chewing gum, a lozenge, or a quick-dissolving tablet.

The compositions described herein may also include additional compounds, such as a binder, a filler, a flavoring agent, a scenting agent, a coloring agent, a preservative, a softening agent, a penetration enhancer, an elastomeric solvent, or mixtures thereof. The penetration enhancers may be of the type that alters the nature of the oral mucosa to enhance penetration, or of the type that alters the nature of the therapeutic agent to enhance penetration through the oral mucosa. Suitable penetration enhancers that may be used with the compositions described herein include polyoxyethylene 23-lauryl ether, aprotin, azone, benzalkonium chloride, cetylpyridinium chloride, cetyltrimethylammonium bromide, cyclodextrin, dextran sulfate, lauric acid, propylene glycol, lysophosphatidylcholine, menthol, methoxysalicylate, methyloleate, oleic acid, phosphatidylcholine, polyoxyethylene, polysorbate 80, sodium ethylenediaminetetraacetic acid (“EDTA”), sodium glycocholate, sodium glycodeeoxycholate, sodium lauryl suflate, sodium salicylate, sodium taurocholate, sodium taurodeoxycholate, as well as certain sulfoxides and glycosides, and mixtures thereof.

I. Therapeutic Agents

A wide variety of different therapeutic agents may be suitable for use with the compositions described herein. For example, the therapeutic agents may be basic, acidic, or amphoteric in nature. In general, the therapeutic agents described herein have an ionized form and an un-ionized form, and are initially present at least partly in an ionized form. As described in more detail below, the buffer system of the described compositions helps to convert substantially all of the therapeutic agent, from its ionized form to its un-ionized form. Therefore, the selection of a suitable therapeutic agent is limited only by the capacity of the agent to be placed in an un-ionized form by the buffer systems described herein.

As used herein, the term therapeutic agent includes all pharmaceutically acceptable forms of the agent being described. For example, the therapeutic agent may be in a racemic or isomeric mixture, or may be a solid complex bound to an ion exchange or the like. In addition, the therapeutic agent may be in a solvated form. The term therapeutic agent is also intended to include all pharmaceutically acceptable derivatives and analogs of the described agent, as well as mixtures of any of the above. The ionized forms of the therapeutic agents include the salt form of the therapeutic agents, for example, a succinate, tartarate, bitartarate, dihydrochloride, salicylate, hemisuccinate, citrate, maleate, hydrochloride, carbamate, sulfate, nitrate, benzoate, mixtures thereof, and the like.

A. Basic Agents

Conversion of the ionized form to the un-ionized form for basic agents is related to pH by the formula: pH=pKa +Log₁₀ (Un-ionized concentration/Ionized concentration). When the pH is the same as the pKa, equimolar concentrations of the unionized form and ionized form exist. For basic agents, when the pH is one unit higher than the pKa, the ratio of the un-ionized form to the ionized form is 91:9. Similarly, when the pH is two units higher than the pKa, the ratio of un-ionized form to the ionized form is 100:1. As noted above, the un-ionized form is lipophilic and, therefore, more capable of passing through the mucous membranes than the ionized form, which is lipophobic in nature. Accordingly, increasing the pH of the saliva favors conversion of the ionized form into the un-ionized form for basic agents, and the final pH may be determined by making use of the above formula for any basic agent.

Any number of basic agents may be selected for use with the compositions described herein. The basic agents may be selected from a wide variety of chemical classes, and be useful in treating a wide variety of indications. For example, illustrative classes from which an acceptable agent may be selected include the opioid compounds, 5-HT₃ receptor antagonist compounds, and 5-HT agonist vasoactive agents.
Opioids

In one variation, the basic agent is selected from among the natural and synthetic opioid compounds. The opioid compounds are thought to be useful in inducing sleep and in relieving pain. Specific examples of opioid compositions that may be used include, but are not limited to, alfentanil, allylprodine, alphaprodine, anileridine, benzylmorphine, bezitramide, clonitazene, codeine, dextromoramide, diampromide, dihydrocodeine, dimenoxadol, dimepheptanol, dimethylthiambutene, dioxaphetyl butyrate, dipipanone, ethoheptazine, ethylmethylthiambutene, ethylmorphine, etonitazene fentanyl, heroin, hydrocodone, isomethadone, levophenacylmorphan, lofentanil, meperidine, methadone, myrophine, narceine, nicomorphine, norlevorphanol, normethadone, norpipanone, opium, oxycodone, papaveretum, phenadoxone, phenoperidine, piminodine, piritramide, propheptazine, promedol, properidine, propiram, propoxyphene, sufentanil, tramadol, tilidine, and mixtures thereof.

Illustrative examples of these basic opiods are oxycodone and its derivatives, each having a N-alkyl piperidine group, as shown below.

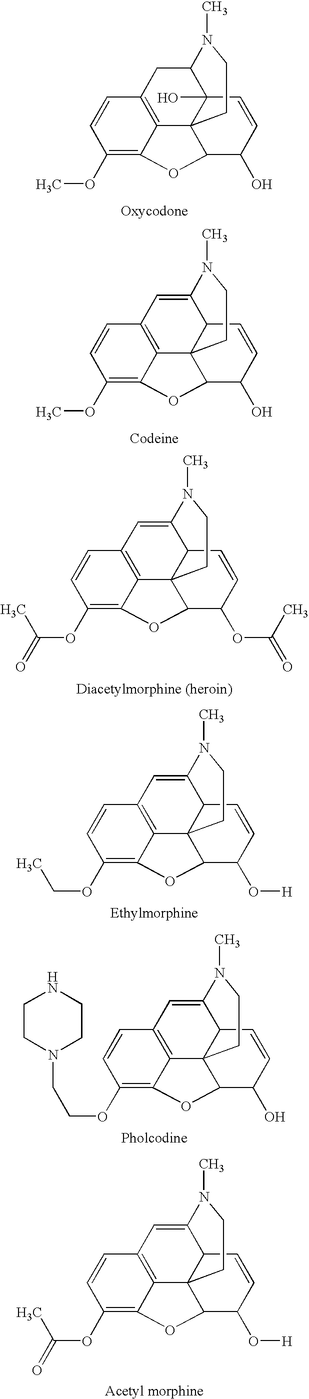

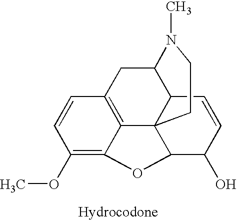

For these opiates, the nitrogen in the N-alkyl piperidine group controls the extent of ionization and the degree of lipophilicity in the given medium. Typically the N-alkyl piperidine group imparts a pKa to the molecule of about 9.5. Therefore, using the above formula, these basic agents are 90% converted to their un-ionized form at a pH of about 10.5.
5-HT₃ Receptor Antagonists

In other variations, the therapeutic agent comprises a 5-HT₃ receptor antagonist compound. These compounds are thought to be useful in the treatment of nausea and vomiting. In general, 5-HT₃ antagonists consist of three main components: (1) an aromatic structure; (2) a carbonyl-containing linking moiety; and (3) an out-of-plane basic nitrogen containing heterocyclic group. These groups have the specific spatial arrangement shown below.

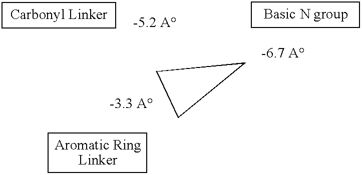

The 5-HT₃ antagonists are able retain their pharmacophore activity by either incorporating the carbonyl linker within the fused rings of the aromatic groups, or by having the carbonyl group directly attached (as a spacer unit) to the aromatic ring and the basic nitrogen group. Those 5-HT₃ antagonists belonging to the former group may be represented by ondansetron. Illustrative examples of therapeutic agents falling within this group are provided in Table 1. Those 5-HT₃ antagonists belonging to the latter group may be represented by granisetron. Illustrative examples of therapeutic agents falling within this group are provided and in Table 2.

TABLE 1
The 5-HT₃ antagonists with carbonyl group incorporated within the fused
aromatic ring.

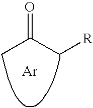

| Drug | Ar | R |
|---|---|---|
| Ondansetron | 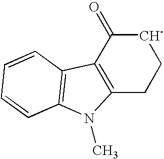 | 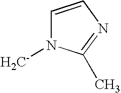 |
| Cilasetron | 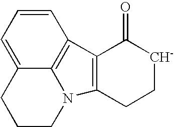 | 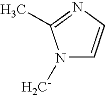 |
| Alosetron | 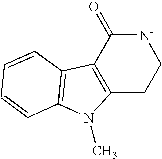 | 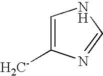 |
| Palonosetron | 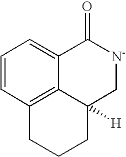 | 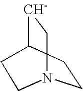 |

TABLE 2
The 5-HT₃ antagonists where the carbonyl group is attached to the
aromatic group and the nitrogen containing basic group as a spacer.

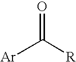

| Compound | Ar | R |
|---|---|---|
| Granisetron | 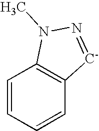 | 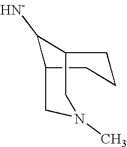 |
| Tropisetron | 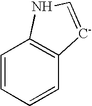 | 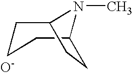 |
| Dolasetron | 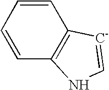 | 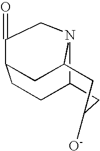 |
| Bernesetron | 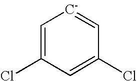 | 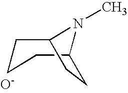 |
| Ramosetron | 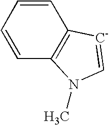 | 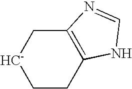 |
| Azasetron | 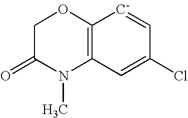 | 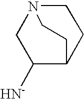 |
| Itasetron | 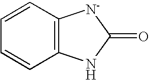 | 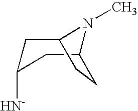 |
| Zacopride | 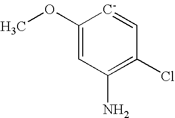 | 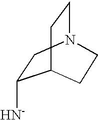 |

As can be seen by the examples provided in Table 1 and Table 2, the constant feature among the 5-HT₃ antagonists is the basic nitrogen group. The basic nitrogen group can be classified generally as imidazole (for example the N in ondansetron), or as a nitrogen-containing heterobicyclic derivative.

Using the above formula for basic agents, the overall lipophilicity and ionization activity of 5-HT₃ antagonists may be controlled and modulated by regulating the pH of the medium containing the 5-HT₃ antagonist agent relative to the pKa of the basic nitrogen group. The imidazole groups, when in conformational vicinity of an electron withdrawing carbonyl group, tend to have pKas in the region of 7.4, and may be converted to their un-ionized, lipophilic form at a pH of about 9.4. In comparison, the 5-HT₃ antagonists that contain nitrogen in a bicyclic ring tend to have a pKa of about 8.8 and thus may be converted to their un-ionized, lipophilic form at a pH of 10.8. As shown in Tables 1 and 2 above, examples of suitable 5-HT₃ receptor antagonist compounds include, but are not limited to ondansetron, palonosetron, tropisetron, lerisetron, alosetron, granisetron, dolasetron, bernesetron, ramosetron, azaseteron, itasetron, zacopride, cilasetron, and any other 5-HT₃ antagonist containing imidazole, oxazole, thiazole, pyrazole, 3-pyrroline or pyrrolidine in their structural formula.
5-HT Agonist Vasoactive Agents

In other variations, the basic agent is selected from the group of 5-HT agonist vasoactive agents. 5-HT agonist vasoactive agents are those agents with selective or non-selective vasoactivity on blood vessels. Examples of illustrative 5-HT agonist vasoactive agents include sumatriptan, zolmitriptan, naratriptan, rizatriptan, eletriptan, almotriptan, frovatriptan, and mixtures thereof.

These agents are indole derivatives useful in the treatment of migraines, which have the following basic indole nucleus.

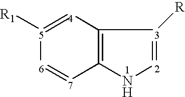

R is typically an alkyl, alkenyl, cycloalkyl, or cycloalkenyl group and R₁ is typically a sulfonamide, an oxazolidinone, a triazole, or a sulfonyl group, any of which may be optionally substituted. A few illustrative agonists are shown below.

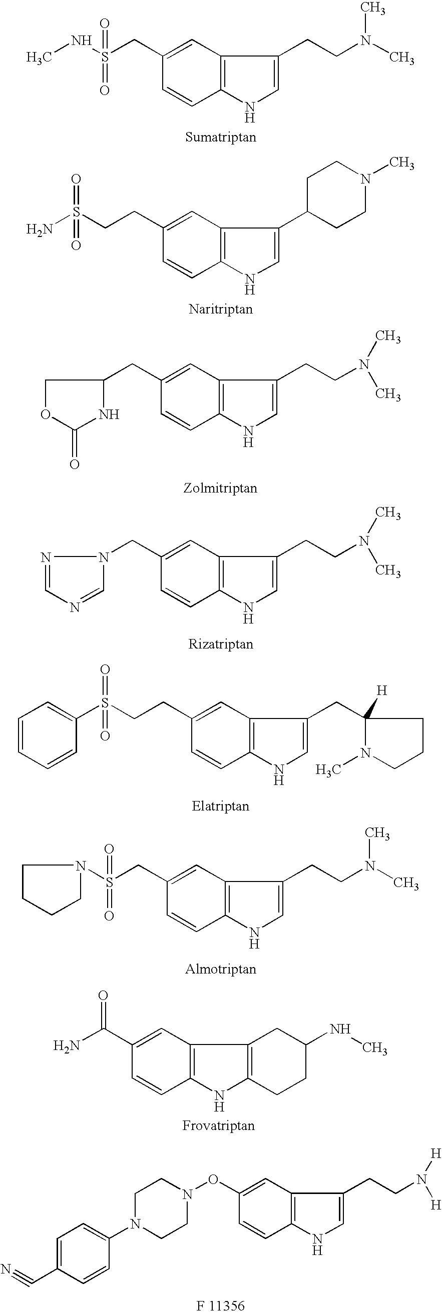

Typically the primary, secondary, or tertiary amines of these agents control the extent of ionization of the molecule.

B. Acidic Agents

Conversion of the ionized form to the un-ionized form for acidic agents is related to pH by the formula: pH=pKa+Log₁₀ (Ionized concentration/Un-ionized concentration). Accordingly, when the pH is the same as the pKa, equimolar concentrations of the un-ionized form and the ionized form exist at that pH. At one pH unit lower than the pKa, the ratio of the un-ionized form to the ionized form for an acidic agent is 91:9. Similarly, at two pH units lower than the pKa, the ratio of the un-ionized form to the ionized form for an acidic agent is about 100:1. Therefore, when the pH is two units lower than the pKa of an acidic agent, the acidic agent exists almost entirely in a lipophillic form. Accordingly, lowering the pH of the saliva favors conversion to the un-ionized form for acidic agents. Any number of acidic agents may be used with the described compositions. One example of a suitable acidic agent is montelukast.

C. Amphoteric Agents

The therapeutic agent may also be amphoteric. Amphoteric agents are those agents having both acidic and basic characteristics. In some variations of the described composition, amphoteric opioids are used. In general, these opioids contain either a phenanthrene nucleus, or something structurally very similar to a phenanthrene nucleus. One example of a suitable amphoteric opioid is morphine, shown below.

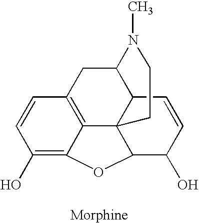

As shown by the structure above, morphine is a polycyclic aromatic compound containing an N-methyl piperidine ring and an oxygen bridge. Other features of the molecule include a phenolic hydroxyl at position 3, an alcohol hydroxyl at position 6 and chiral carbons at positions 5, 6, 9, 11 and 13. The simultaneous presence of the cationic N-methyl piperidine ring and the anionic phenolic hydroxyl group renders the opioid amphoteric. In comparison to the basic opioids, the extent of ionization for amphoteric opioids is modulated not by the pKa of the individual functional group but by the isoelectric point of the molecule. The isoelectric point is the pH at which the net charge on the molecule is zero and the compound is most permeable. The isoelectric point of morphine is 9.1.

The structural similarity between various opioids and their dependence on similar functional groups for analgesic activity can be investigated using morphine. A relatively simple modification of morphine's phenanthrene nucleus significantly alters the pharmacological function of morphine without affecting the amphoteric nature of the drug or its ability to form zwitterions at its isoelectric point. For example, reacting morphine with a strong mineral acid removes its oxygen bridge and results in the formation of apomorphine, an agent that lacks analgesic activity but has emetic properties. Conversely, replacing the methyl group on the nitrogen of the piperidine ring with an allyl, propyl or cyclopropylmethyl group produces derivatives having opioid antagonistic actions. Some antagonists like nalorphine retain some of their analgesic actions (mixed agonist-antagonist), while others, like naloxone and naltrexone are pure antagonists with no detectable opioid agonistic actions.

Examples of suitable amphoteric agents include, but are not limited to, buprenorphine, butorphanol, cyclazocine, desomorphine, dezocine, dihydromorphine, eptazocine, hydromorphone, hydroxypethidine, ketobemidone, levallorphan, levorphanol, meptazinol, metazocine, metopon, morphine, nalbuphine, nalorphine, naloxone, naltrexone, normorphine, oxymorphone, pentazocine, phenomorphan, phenazocine, and mixtures thereof. A few of these are shown below.

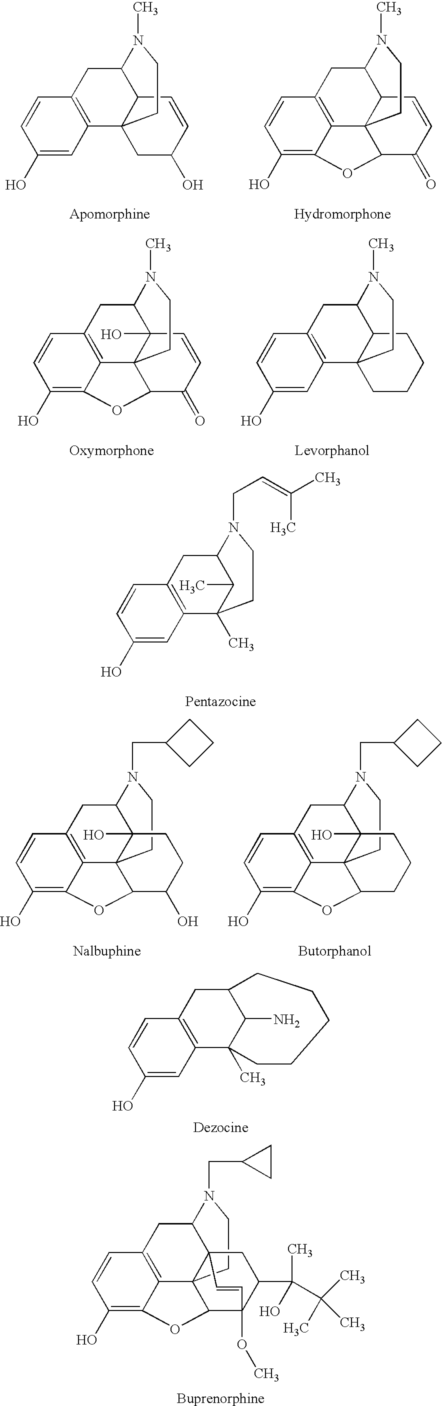

II. Buffer System

In general, the buffer systems of the compositions described herein are capable of changing the pH of saliva from an arbitrary initial pH to a predetermined final pH, independent of the arbitrary initial pH, and of sustaining the predetermined final pH for a period of time. The formulas provided above are used to determine the final pH at a given extent of ionization. The final pH is therefore dependent upon the nature of the therapeutic agent (i.e., basic, acidic, or amphoteric), upon the pKa of the agent or its isoelectric point (for amphoteric agents), and upon the desired extent of conversion from the ionized form to the un-ionized form.

As noted above, the final pH is typically determined when substantial conversion (e.g., greater than 50%, 80%, 90, or 95%) to the un-ionized form is assumed. In this way the buffer system favors substantial conversion to the un-ionized form when used with the described compositions in vivo. Once the final pH has been determined using the above formulas, buffering agents may then be selected to achieve that pH.

The buffer system comprises at least two different buffering agents, but any number of buffering agents may be used as practicable, so long as the buffering system achieves the final pH, determined as described above. Similarly, a wide variety of different buffering agents may be used. In general, when a binary buffer system is used, the buffering agents comprise either a weak acid and a salt of a weak acid, or a base and a salt of the base. Illustrative examples of suitable buffering agents useful in either raising or lowering the pH of saliva include sodium carbonate, sodium bicarbonate, potassium carbonate, potassium bicarbonate, potassium citrate and mono basic potassium phosphate, magnesium oxide, magnesium carbonate, magnesium bicarbonate, alkaline starch, ascorbic acid, and mixtures thereof.

The concentrations of the buffering agents are tailored such that the predetermined final pH of the saliva is achieved and sustained for a period of time, for example, at least 5 minutes, at least 10 minutes, or at least 20 minutes. This typically involves a trial and error type of procedure of adding various amounts of buffering agents and then measuring the final pH over time. In this way selection of an appropriate weight ratio for the given buffering agents may be easily determined in just a few trials. For binary systems, typically the buffering agents are in a weight ratio of from about 1-10:10-1. More typically, for binary systems, the buffering agents are in a weight ratio range of from about 1-2:2-1, of about 1-3:3-1, of about 1-5:5-1. In some instances, the buffering agents are in a 1:1 ratio by weight for binary systems. Similar modifications may be made for tertiary or quaternary buffer systems, and the like.

In the case where acidic therapeutic agents are used, the buffer system lowers the pH of the saliva. In these variations, the final pH of saliva is typically in the range of 1-6.9, and more typically 2-4. In the case where basic therapeutic agents are used, the buffer system raises the pH of the saliva. In these variations, the final pH of saliva is typically in the range of 7.1-13, and more typically, 9-11. The final pH for amphoteric agents is dependent upon the isoelectric point of the molecule as described above, and therefore, the buffer system may either raise or lower the pH of saliva, depending upon the particular amphoteric agent chosen. In each instance, however, the final pH of saliva is typically of such a value that no damage is caused to the oral cavity or the mucous membranes. For example, a pH below 2 and a pH above 11.5 may be undesirable. Because of the limitations due to toxicity, it will be understood that in some situations, the extent of conversion to the un-ionized form may not reach 100%.

In addition to altering the pH of saliva, the buffer systems described herein may have the further advantage of altering the taste characteristics of the composition. This may be quite desirable when therapeutic agents, which are very bitter, are chosen for transmucosal delivery. Typically, as the pH is lowered, the taste of the final composition becomes less bitter.

While the foregoing discussion has focused on the ability of the buffer system to alter saliva pH to favor substantial conversion to the un-ionized form of a therapeutic agent, it is conceivable that the buffer system may have subsidiary beneficial effects on the extent of absorption as well. For example, the buffer system may create a final saliva pH that in turn effects the molecular configuration of the therapeutic agent in a way in which absorption is increased. It is to be understood that these subsidiary beneficial effects of the buffer system are within the general scope of the buffer system and compositions herein described.

III. Dosage Forms

While each individual possesses unique factors that may affect the rate and extent of absorption of the therapeutic agents described herein, each of the described dosage forms offer advantages over the traditional dosage forms for oral administration. For example, each of the below dosage forms avoids hepatic first pass metabolism, degradation within the GI tract, and drug loss during absorption. Consequently, the amount of therapeutic agent required per dose is less than would be required if formulated, for example, in a pill for oral administration. Similarly, with each of the below dosage forms, the bioavailability of the active is increased, and hence the time to onset of therapeutic activity is reduced.
A. Chewing Gum

In some variations, the final dosage form for the described compositions is in the form of a chewing gum. In general, the chewing gum compositions comprise a therapeutic agent, a gum base, a protecting agent, and a buffer system. Suitable therapeutic agents and buffer systems were discussed in detail above. The percentage of therapeutic agent in the chewing gum composition will vary depending upon the specific therapeutic agent selected. Similarly, the percentage of buffering agents will vary depending upon the specific therapeutic agent and buffering agents selected.
Gum Base

In general, the gum base comprises a material selected from among the many water- and saliva-insoluble gum base materials known in the art. In some variations, the gum base is comprises at least one hydrophobic polymer and at least one hydrophilic polymer. Illustrative examples of suitable polymers for gum bases include both natural and synthetic elastomers and rubbers, as well as mixtures thereof. Examples of suitable natural polymers include, but are not limited to, substances of plant origin like chicle, jelutong, gutta percha and crown gum. Examples of suitable synthetic elastomers include butadiene-styrene copolymers, isobutylene and isoprene copolymers (e.g., “butyl rubber”), polyethylene, polyisobutylene, polyvinylesters, such as polyvinyl acetate and polyvinyl acetate phthalate, and mixtures of any of the foregoing. In some variations, the gum base comprises a mixture of butyl rubber (a copolymer of isoprene and isobutylene), and polyisobutylene, and optionally, polyvinylacetate (preferably PVA having a MW of approximately 12,000).

Typically the gum base comprises from about 25% to about 75% of such polymers, and more typically, from about 30% to about 60%. Unless otherwise stated, all percentages provided herein are weight percentages, based on either the total weight of the gum base or of the final chewing gum composition, where noted.

The gum base may also include additional compounds, such as plasticizers (e.g., softeners or emulsifiers). These compounds may, for example, help reduce the viscosity of the gum base to a desirable consistency and improve its overall texture and bite. These compounds may also help to facilitate release of the active upon mastication. Non-limiting examples of these compounds include, lecithin, mono- and diglycerides, lanolin, stearic acid, sodium stearate, potassium stearate, glycerol triacetate, glycerol monostearate, glycerin, and mixtures thereof. The gum base typically comprises from about 0% to about 20% of plasticizer compounds, and more typically from about 5% to about 15%.

The gum base may further comprise waxes, such as beeswax and microcrystalline wax, and fats or oils, such as soybean and cottonseed oil. Typically, the gum base comprises from about 0% to about 25% of these waxes and oils, and more typically will comprise from about 15% to about 20%.

The gum base may further comprise one or more elastomeric solvents, for example, rosins and resins. Illustrative examples of such solvents include methyl, glycerol, and pentaerythritol esters of rosins or modified rosins, such as hydrogenated, dimerized or polymerized rosins or mixtures thereof (e.g., pentaerythritol ester of partially hydrogenated wood rosin, pentaerythritol ester of wood rosin, glycerol ester of wood rosin, glycerol ester of partially dimerized rosin, glycerol ester of polymerized rosin, glycerol ester of tall oil rosin, glycerol ester of wood rosin and partially hydrogenated wood rosin and partially hydrogenated methyl ester of rosin, such as polymers of alpha-pinene or beta-pinene, and terpene resins including polyterpene and mixtures thereof). Typically the gum base comprises from about 0% to about 75% of an elastomeric solvent, and more typically less than 10%.

The gum base may further comprise a filler material to enhance the chewability of the final chewing gum composition. Fillers that are substantially non-reactive with other components of the final chewing gum formulation are desirable. Examples of suitable fillers include calcium carbonate, magnesium silicate (talc), dicalcium phosphate, metallic mineral salts (e.g., alumina, aluminum hydroxide, and aluminum silicates), and mixtures thereof. Typically, the gum base comprises about 0% to about 30% of a filler, and more typically about 10% to about 20%.

The gum base may further comprise a preservative, such as butylated hydroxy toluene (“BHT”), and the like. Typically, the gum base comprises only trace amounts of a preservative, for example, less than about 0.1%.

The total chewing gum composition typically comprises from about 20% to about 90% of gum base, more typically less than about 70%, and most typically, from about 50% to about 60% of gum base. In certain instances the use of too much gum base may interfere with the release of the active ingredient, and additionally, may contribute to tackiness and poor mouth-feel of the final product. The use of a protecting agent, as described below may help to ameliorate this effect.

The chewing gum composition may further comprise at least one bulk sweetener to improve the palatability of the composition by masking any unpleasant tastes it may have. The sweetener may be incorporated into the gum base, but need not be. Examples of suitable sweeteners include compounds selected from the saccharide family, such as the mono-, di-, tri-, poly-, and oligosaccharides; sugars, such as sucrose, glucose (corn syrup), dextrose, invert sugar, fructose, maltodextrin, polydextrose; saccharin and its various salts, such as the sodium and calcium salts, cyclamic acid and its various salts; dipeptide sweeteners; chlorinated sugar derivatives such as sucralose, dihydrochalcone; and sugar alcohols such as sorbitol, sorbitol syrup, mannitol, xylitol, hexa-resorcinol and the like, including mixtures thereof. Hydrogenated starch hydrolysate, and the potassium, calcium and sodium salts of 3,6-dihydro-6-methyl-1-1,2,3-oxathiazin-4-one-2,2-dioxide may also be used. Of the foregoing, sorbitol, mannitol, and xylitol, either alone or in combination are most typically used. The chewing gum composition comprises typically comprises from about 5% to about 75% of the sweetener, more typically from about 25% to about 40%, and most typically from about 30% to about 35%.

The chewing gum composition may further comprise one or more flavoring agents. The flavoring agent may be natural, synthetic, or a combination thereof. Examples of suitable flavoring agents include peppermint, spearmint, wintergreen, cinnamon, menthol, cherry, strawberry, watermelon, grape, banana, peach, pineapple, apricot, pear, raspberry, lemon, grapefruit, orange, plum, apple, fruit punch, passion fruit, mixtures thereof, and the like. Coloring agents (natural, artificial, or combination thereof) may also be used. The coloring agents may also be used to color code the composition, for example, to indicate the type and dosage of the therapeutic agent therein. Suitable coloring agents include FD & C coloring agents, natural juice concentrates, pigments such as titanium oxide, silicon dioxide, and zinc oxide, and the like. The chewing gum composition typically comprises from about 0% to about 10% of the flavoring and coloring agents, either alone or in combination. More typically, the chewing gum composition comprises from about 0.1% to about 5% of the flavoring and coloring agents, and even more typically, from about 2% to about 3%.

The gum base need not be prepared from its individual components. The gum base may be purchased with the desired ingredients therein, and may or may not be modified. Several manufacturers produce and gum bases, which may be suitable for use with the described chewing gum compositions. Examples of such suitable gum bases are the Pharmgum™ M, S, or C, sold by SPI Pharma Group in New Castle, Del. In general, the Pharmagums are a mixture of gum base, sweetener, plasticizer, and sugar. Literature on Pharmagum is readily available from its manufacturer, SPI Pharma Group, 321 Cherry Lane, New Castle, Del. 19720-2780.

In some variations, the chewing gum composition includes a therapeutic agent centerfill. A centerfill may be particularly suitable when immediate release of the therapeutic agent is especially desirable. In addition, encapsulating the therapeutic agent in a centerfill may help to mask any undesirable taste the therapeutic agent may have.

In these variations, the gum base surrounds, at least in part, a centerfill. The centerfill comprises at least one therapeutic agent, and may be a liquid or semi-liquid material. In some variations, the centerfill material may be low-fat or fat free. The centerfill may also contain one or more sweeteners, flavoring agents, coloring agents, and scenting agents as described herein. In some variations, the centerfill further includes a buffer system as described herein. In one variation, the centerfill comprises a combination of saccharide material, flavoring agent, a polyol, and an edible gel material.
Protecting Agent

The chewing gum composition further comprises a protecting agent. The protecting agent coats at least part of the therapeutic agent, typically upon the mixing of the two agents. The protecting agent may be mixed with the active in a ratio of about 0.1 to about 100 by weight, more typically in a ratio of about 1 to about 50 and most typically in a ratio of about 1 to about 10.

The protecting agent reduces the adhesion between the therapeutic agent and the gum base so that the therapeutic agent may be more easily released from the gum base. In this way, the therapeutic agent may be delivered across the mucous membranes in about 5 to about 20 minutes of chewing, and desirably within about 10 minutes of chewing. A variety of different protecting agents may be used. Examples of suitable protecting agents include calcium stearate, glycerin monostearate, glyceryl behenate, glyceryl palmitostearate, hydrogenated castor oil, hydrogenated vegetable oil type I, light mineral oil, magnesium lauryl sulfate, magnesium stearate, mineral oil, poloxamer, polyethylene gycol, sodium benzoate, sodium chloride, sodium lauryl sulfate, stearic acid, cab-o-sil, talc, zinc stearate, and mixtures thereof.
Methods of Making Chewing Gum Compositions

In some variations, the chewing gum composition is made from gum base granules. In these variations, the gum base takes the form of granules, with the therapeutic agent interspersed among the granules. The gum base granules together with the therapeutic agent are then compressed together to yield the final formulation.

In these variations, the chewing gum composition may be prepared using the procedures set forth in U.S. Pat. No. 4,405,647, which is hereby incorporated by reference in its entirety. According to the procedures set forth therein, the gum base material may be melted or softened using one or more of the softening agents, plasticizers and/or solvent and filler materials as described above. The sweeteners and flavoring agents are then mixed into the gum base by comminuting the gum base material together with the water-soluble ingredients in a bed or blender within a gaseous medium at approximately 25° C. The resultant material is then continuously pulverized and chopped into smaller particles. To prevent adherence of the resultant particles to one another, filler or bulking material may be added, such as lubricants, glidants or any other tableting or compression aid, such as silica gel or calcium carbonate. Granules of any desired size and shape may be obtained when a standard mesh screen is selected to separate them.

The therapeutic agent is then prepared to be mixed with the formed particulates such that the active is released within 20 minutes of chewing, and more desirably, within 5 to 15 minutes, and most desirably within 10 minutes. This is done by mixing the therapeutic agent with a protecting agent as described above. Once the therapeutic agent is coated at least in part by the protecting agent, gum base is then continuously mixed into the therapeutic agent/protecting agent mixture. During this mixing procedure, the therapeutic agent/protecting agent (and gum base, if already added) mixture is typically always in an excess amount relative to the incoming, or newly added, gum base. In this way, the newly added gum base is diluted by the therapeutic agent/protecting agent and gum base mixture.

Upon thorough mixing (using any suitable device), the materials are then compressed and compacted in a tablet press, or the like. In this way the therapeutic agent is sandwiched in the voids between the compressed particulate gum granulate material and vice versa. The therapeutic agent is thus made “external” to the gum base material itself. In one variation, the therapeutic agent together with the particulates, heretofore described, are provided in a substantially non-liquid format. That is, the formulation of the invention according to this embodiment is preferably substantially 0% liquid.

In some variations, the gum base granules are further mixed together with the buffer system described herein. In other variations, the gum base granules are mixed with a single buffering agent, with at least one additional buffering agent being mixed with the therapeutic agent. In still other variations, the gum base granules are mixed with half of the buffer system, with the other half being mixed with the therapeutic agent.

The variations of the chewing gum compositions comprising a centerfill may be prepared using methods known in the confectionery and chewing gum industries. For example, U.S. Pat. No. 3,806,290, which is hereby incorporated by reference in its entirety, describes a method for forming a centerfill chewing gum by extruding a hollow-centered rope of chewing gum through an orifice having a pair of concentric conduits extending therethrough. A centerfill material is fed through the inner conduit to the hollow center upstream through a space between the inner and outer conduits. The centerfill rope of chewing gum is then passed to a sizing unit having a plurality of pairs of rollers for progressively decreasing a cross-sectional dimension of the gum rope. The plurality of pairs of rollers includes at least one vertical pair of rollers having vertically aligned axes of rotation and overlapping lower flange portions. A ramp structure is provided for guiding the gum rope above the roller flange portions upon entry of the gum rope between the vertical pair of rollers, to produce the final gum. Other methods of forming centerfill chewing gum known in the art may also be utilized.

The chewing gum compositions can have any desired shape, size, and texture. For example, the composition may have the shape of a stick, tab, gumball, and the like. Similarly, the gum may be any desirable color. For example the gum may be any shade of red, blue, green, orange, yellow, violet, indigo, and mixtures thereof, and may be color coded as described above. The gum can be individually wrapped or grouped together in pieces for packaging by methods well known in the art.

B. Lozenges and Candy

In some variations, the final dosage will have the form of a lozenge or a candy. These types of dosage forms are held in the mouth, and are slowly dissolved by the user's saliva. Any type of lozenge or candy, having any number of desirable shapes and sizes may be used with the compositions described herein. A general discussion of lozenges and candies is provided in H. A. Lieberman, Pharmaceutical Dosage Forms, Volume 1: Tablets (1989), Marcel Dekker, Inc., New York, N.Y. at Medicated Confections, pages 75-418, which is hereby incorporated by reference in its entirety.

In general, the lozenge or candy comprises a therapeutic agent, a carrier, and a buffer system. The therapeutic agent and the buffer system are described in detail above. The carrier differs from that of the above described chewing gum compositions in that the water insoluble gum base is typically replaced by a water soluble natural or synthetic gum or binder. For example, in some variations, a suitable lozenge may be formed by replacing the Pharmagum described above with gum acacia or other appropriate gums or binders. The lozenges may optionally comprise diluents, disintegrators, flavoring agents, coloring agents, and scenting agents.

C. Quick-dissolving Tablets

As indicated by their name, quick-dissolving tablets dissolve quickly after being placed within the mouth of a user. The tablet is dissolved by the user's saliva, without the need for chewing. This type of dosage form may be particularly desirable for pediatric and geriatric patients, since small children and aged individuals often have difficulty in chewing items.

In general, the quick-dissolving tablets comprise a therapeutic agent, a carrier, and a buffer system. As with the chewing gum compositions and lozenges described above, any number of flavoring agents or scenting agents may also be employed. Suitable therapeutic agents and buffer systems are described above. The carrier of the quick-dissolving tablet is typically a binder compound that is useful in keeping the tablet in a semi-solid state, and may be a solid, or a liquid, and may for example be a high-melting point fat or waxy material. Materials suitable as binders are discussed in detail above and may be used alone, or in combination, with the quick-dissolving tablets described here. The quick-dissolving tablets may be of any desirable shape, size, or color as described above.

### EXAMPLES

The following examples are provided only to demonstrate various aspects of the compositions and methods described herein. It is to be understood that these example are not comprehensive or exhaustive of the many variations of the compositions herein described. These examples are non-limiting, and for illustrative purposes only.

A. Ondansetron
Membrane Assay

The effect of pH adjustment on the extent of ionization, and hence, the extent to which a therapeutic agent will traverse the mucous membranes may be demonstrated by a membrane assay, see Kansy, M., Senner, F., Gubernator, K., 1998. “Physicochemical high throughput screening: parallel artificial membrane assay in the description of passive absorption processes.” J. Med. Chem., 41, 1007-1010; and Avdeef, “A. Physicochemical profiling (solubility, permeability, and charge state).” Curr. Topies Med. Chem. 2001, 1, 277-351. This assay uses a lipid-coated membrane to predict lipid mucosal membrane penetration.

The membrane apparatus consists of a dodecane membrane sandwiched between a donor and acceptor cell. The lipid-coated membrane is less porous then the mucous membranes of the oral cavity. Thus, the enhancement seen in the membrane assay is very likely to be magnified in vivo.

Membrane assays were performed using ondansetron HCl solutions at a pH of 5.4, 7.4 and 8.5. The alkaline pH values of 7.4 and 8.5 were adjusted using freshly prepared 0.01 M sodium bicarbonate—sodium carbonate buffer solution. The acidic pH of 5.4 was achieved using a 0.01 M acetate (sodium acetate & acetic acid) buffer solution. Permeation through the membrane was measured by determining the concentrations of ondansetron in the acceptor cell and is expressed as Pₑ (effective permeability in centimeters per second).

The results shown in Table 3 below demonstrate that the effective permeability of ondansetron HCl increases by more than 200% at a pH of 8.5 relative to the corresponding pH of 7.4 and 1000% relative to the pH of 5.4.

TABLE 3
Effective Permeability of Ondansetron in Membrane Assay.

| pH | Pₑ (cm/s) |
|---|---|
| 5.4 | 0.32 |
| 7.4 | 1.30 |
| 8.5 | 3.25 |

The ondansetron may be formulated as a chewing gum composition as described above. In these variations, the unit dose, or serving for the chewing gum composition comprises about 0.1 to about 100 milligrams of ondansetron (as measured in its free base form), more desirably, from about 1 to about 50 milligrams, and most desirably from about 2 to about 25 milligrams. In some variations, it may be particularly desirable to include about 2-5 milligrams of ondansetron per serving, with perhaps 4 milligrams being especially desirable. Extra ondansetron, up to about 10-25% or so by weight may be added as “overage” or, the amount that may be expected to be “washed away” and not otherwise released or absorbed during mastication.

Given in weight percentages, the total amount of ondansetron (in whatever chosen form, measured as per its free base form) will typically be in the range of about 0.01% to about 10%, more typically from about 0.05% to about 2.0%, and most typically, from about 0.1% to about 1.0%. In some variations, 0.25% is particularly desirable. The foregoing percentages will vary depending upon the particular source of ondansetron, the amount of ondansetron desired in the final formulation, and on the desired release rate of the ondansetron.

The buffer system of the ondansetron chewing gum composition should result in a final salivary pH in excess of at least about 7.5, and even more desirably in the range from about 8 to about 10. A pH level of at least about 9.5 is most desirable.

The ondansetron chewing gum containing the corresponding buffer system can be used for treatment of emesis, caused by a variety of clinical and pathological reasons, particularly the nausea and vomiting associated with cancer chemotherapy and radiotherapy, see, Green et al., Cancer Chemother. Pharmacol., 24:137-139 (1989). After introduction of a serving size piece of the gum composition into the mouth, the consumer will chew the gum as is normally done with any non-medicated type of chewing gum for about 20-30 minutes, but at approximately an average rate of about 10-45 chews per minute. The gum is then discarded.

A serving of the ondansetron chewing gum delivery system is typically designed to cause a loaded ondansetron concentration level in the bloodstream of at least about 10 to 300 nanograms of ondansetron per milliliter of plasma. For example, a 24 mg ondansetron chewing gum may be designed to produce a mean peak plasma concentration within the range of 150 to 300 nanograms of ondansetron per milliliter of plasma in 5 minutes to 2 hours. Similarly, an 8 mg dose may be designed to produce a mean peak plasma concentration within the range of 25 to 100 nanograms of ondansetron per milliliter of plasma in 5 minutes to 2 hours.

C. Oxycodone
Membrane Assay

The dissociation constant (pKa) of oxycodone is 8.9, and therefore the drug would be 100% un-ionized at pH 10.9 and 99% at pH 9.9. Membrane assays were performed using oxycodone HCl solutions at a pH of 6.5, 9.5, and 10.0. The pH of the solutions was adjusted using a freshly prepared 0.01 M sodium bicarbonate—sodium carbonate buffer. Permeation was measured by determining the concentrations of oxycodone in the acceptor cell and is expressed as Pₑ (effective permeability in centimeters per second).

The results shown below in Table 4 demonstrate how the effective permeability of oxycodone HCl increases with pH.

TABLE 4
Effective Permeability of Oxycodone in Membrane Assay.

| pH | Pₑ (μm/s) |
|---|---|
| 6.5 | 2.27 |
| 9.5 | 4.90 |
| 10.0 | 5.54 |

The Area Under the Curve (AUC) values and the plasma concentration (C) for oxycodone (10 mg) were simulated for 0.5 hour and 1 hour after transmucosal administration and compared to those values predicted for the oral administration dose equivalent traditional tablet. These results are shown in Table 5 below.

TABLE 5
Comparison of compositions for delivery across oral mucosa
to traditional oral tablet dosage form.

| | AUC (0-0.5 hr) | C 0.5 hr | AUC (0-1 hr) | C 1 hr |
|---|---|---|---|---|
| Formulation | ng · hr/ml | ng/ml | ng · hr/ml | ng/ml |
| Transmucosal | 2.6 | 10.07 | 6.48 | 11.12 |
| Formulation | | | | |
| Traditional | 0.8 | 4.03 | 4.40 | 8.97 |
| Oral Tablet | | | | |

Chewing Gum

The oxycodone may be formulated as a chewing gum composition as described above. In these variations, the unit dose, or serving for the chewing gum composition comprises from about 0.1 to about 100 milligrams of oxycodone (as measured in its free base form), more desirably, from about 1 to about 50 milligrams, and even more desirably, from about 2 to about 25 milligrams. In some variations, it may be particularly desirable to include about 2-5 milligrams of oxycodone in a serving, with 4 milligrams being especially desirable. Extra oxycodone, up to about 10-25% or so by weight. may be added as “overage.”

Given in weight percentages, the total amount of oxycodone (in whatever chosen form, measured as per its free base form) will typically comprise about 0.01% to about 10%, more typically from about 0.05% to about 2.0%, and most typically, from about 0.1% to about 1.0% of the chewing gum. In some variations, 0.25% oxycodone is particularly desirable. The foregoing percentages will vary depending upon the particular source of oxycodone, the amount of oxycodone desired in the final formulation, and on the desired release rate of the oxycodone.

The buffer system of the oxycodone chewing gum composition provides for a final salivary pH in excess of at least about 7.5, and even more desirably in the range from 8.0 to 11. In some variations, having a pH of at least about 9.5 is most desirable.

The oxycodone chewing gum containing the corresponding buffer system can be used for treatment of pain, caused by a variety of clinical and pathological reasons, for example, to help alleviate the pain associated with cancer. After introduction of a serving size piece of the gum composition into the mouth, the consumer will chew the gum as is normally done with any non-medicated type of chewing gum for about 20-30 minutes, but at approximately an average rate of about 10-45 chews per minute. The gum is then discarded.

A serving of the oxycodone chewing gum is typically designed to cause a loaded oxycodone concentration level in the bloodstream of at least about 10 to 300 nanograms of oxycodone per milliliter of plasma. For example, a 24 mg oxycodone chewing gum may be designed to produce a mean peak plasma concentration within the range of 150 to 300 nanograms of oxycodone per milliliter of plasma in 5 minutes to 2 hours. Similarly, an 8 mg dose may be designed to produce a mean peak plasma concentration within the range of 25 to 100 nanograms of oxycodone per milliliter of plasma in 5 minutes to 2 hours.

D. Sumatriptan
Membrane Assay

The above described assay method may was used to demonstrate the beneficial effects of pH adjustment on membrane penetration for a sumatriptan dosage form. The dissociation constant (pKa) of sumatriptan is 9.5, and therefore the drug would be 100% un-ionized at pH 11.5 and 99% at pH 10.5. Membrane assays were performed using sumatriptan succinate at pH values of 9.0, 9.5, and 10.0. The final pH values of these solutions were adjusted using a freshly prepared 0.01 M sodium bicarbonate—sodium carbonate buffer. Permeation was measured by determining the concentrations of sumatriptan in the acceptor cell, and is expressed as Pₑ (effective permeability in centimeters per second).

As shown in Table 6 below, the effective permeability of sumatriptan increases with pH.

TABLE 6
Effective Permeability of Sumatriptan in Membrane Assay.

| pH | Pₑ (μm/s) |
|---|---|
| 9.0 | 5.99 |
| 9.5 | 9.45 |
| 10.0 | 15.61 |

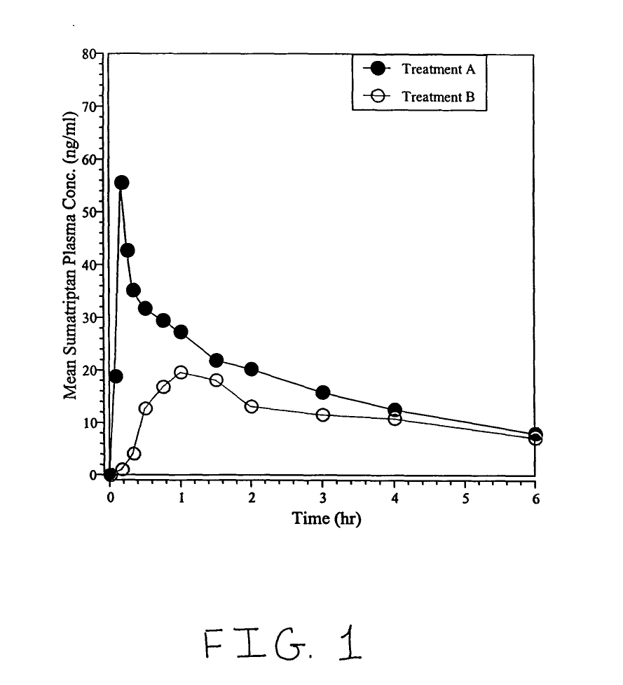

Once again the lipid coated membrane is less porous then the mucosal membrane in the oral cavity. Thus, enhancement will likely be magnified in situ resulting in enhanced buccal absorption and therefore higher bioavailability relative to a dose equivalent traditional oral tablet, as demonstrated for sumatriptan by FIG. 1. That is, FIG. 1 is a graph showing plasma concentration comparisons over time between a transmucosally delivered sumatriptan solution at pH 10 (Treatment A), and an orally administered sumatriptan tablet (Treatment B).
Chewing Gum

The sumatriptan may be formulated as a chewing gum composition as described above. In these varations, the unit dose, or serving for the chewing gum comprises about 0.1 to about 100 milligrams of sumatriptan (as measured in its free base form), more desirably, from about 1 to about 50 milligrams, and even more desirably, from about 2 to about 25 milligrams. In some variations, it may be particularly desirable to include about 2-20 milligrams of sumatriptan in a serving, with 12.5 milligrams being especially desirable. Extra sumatriptan, up to about 10-25% or so by weight may be added as “overage.”

Given in weight percentages, the chewing gum composition typically comprises from about 0.001% to 2.0% of sumatriptan (in whatever chosen form, measured as per its free base form), and more typically from about 0.002% to about 1.0%. In some variations, about 0.008% sumatriptan is used. The foregoing percentages will vary depending upon the particular source of sumatriptan utilized, the amount of sumatriptan the skilled artisan desires to include in the final formulation, as well as on the particular release rate of the sumatriptan desired.

The buffer system of the sumatriptan chewing gum composition provides for a final salivary pH in excess of at least about 7.5, and even more desirably in the range from 8.0 to 11. A pH level of at least about 9.5 is most desirable.

The sumatriptan chewing gum containing the corresponding buffer system can be used for treatment of migraine. After introduction of a serving size piece of the gum composition into the mouth, the consumer will chew the gum as is normally done with any non-medicated type of chewing gum for about 20-30 minutes, but at approximately an average rate of about 10-45 chews per minute. The gum is then discarded.

A serving of the sumatriptan chewing gum is typically designed to cause a loaded sumatriptan concentration level in the bloodstream of at least about 5 to 300 nanograms of sumatriptan per milliliter of plasma. The ratio of the maximum plasma concentration (Cmax) to the time to achieve that maximum plasma concentration (Tmax) is typically within a range of about 10 ng/ml×hr to about 1000 ng/ml×hr, and more typically within a range of about 100 ng/ml×hr to about 500 ng/ml×hr.

A sumatriptan chewing gum was made using the above procedures as follows. Silicon dioxide USP (0.35 kg) was passed through a #20 mesh screen, and then loaded into a blender containing 0.810 kg mannitol granular USP and 9.430 kg Pharmagum C. The material was blended for 10 minutes. Sumatriptan succintate EP (0.173 kg) was ground with the silicon dioxide (0.02 kg) using a mortar and pestle. The remaining silicon dioxide, along with 0.228 kg magnesium stearate was added into the mortar while continuing to grind. The ground materials were transferred into a plastic bag, and the mortar was rinsed using 0.01 kg silicone dioxide, and transferred into the bag. The contents of the bag were then blended for five minutes.

Equal parts of the blended bag contents and the blended mannitol gum base mixture were blended for an additional five minutes. This process was repeated unto all the sumatriptan and gum base mixture had been blended together. Sodium carbonate (0.110 kg), sodium bicarbonate (0.570 kg), gum acacia (0.43 kg), xanthan gum (0.013 kg), aspartame (0.072 kg), were then loaded into the blender with natural and artificial flavors and blended for ten minutes with 0.090 kg of silicon dioxide. The flavors used were as follows natural and artificial grape flavor S.D. (0.215 kg), natural and artificial cherry flavor (0.108 kg), natural and artificial fruit punch flavor S.D. (0.180 kg), natural cherry WONF DURAROME® flavor (0.215 kg), and natural passion fruit type DURAROME® flavor (0.035 kg).

The blend was passed through a #12 mesh screen and then blended for an additional 15 minutes. Magnesium stearate (0.114 kg) was passed through a #20 mesh screen and added to the blend and blended for five minutes. The blend was collected and placed in plastic bags. Two silica gel desiccant bags were placed around the plastic bags to absorb ambient moisture. The blend was then compressed into tablets.

Sumatriptan Quick-Dissolving Tablet

A sumatriptan quick-dissolving tablet was made using the above described processes. Mannitol (3.633 kg) and sorbitol (0.330 kg) were blended for ten minutes. Sodium carbonate (0.330 kg), sodium bicarbonate (0.165 kg), natural peppermint flavor (0.125 kg), natural menthol flavor (0.025 kg), and sucralose (0.020 kg) were blended separately for ten minutes. Stearic acid (0.125 kg), magnesium stearate (0.075 kg), and sumatriptan succinate (0.172 kg) were blended for ten minutes and then passed through a #12 mesh screen. The blended mixtures were then added together and compressed into tablets.

The described compositions provide a convenient, reliable, practical, and painless system for delivering therapeutic agents across the oral mucosa. Notably, the described compositions are capable of rapidly delivering a therapeutic agent so that a pharmacologically effective concentration of the agent enters the bloodstream within 20 minutes, 10 minutes, or even within 1-2 minutes after the therapeutic agent is released from the carrier. Although the invention has been described with respect to certain variations, those of ordinary skill in the art may make modifications without departing from the scope and spirit of the invention.

1. A composition for delivering a therapeutic agent across the oral mucosa comprising:
at least one therapeutic agent at least partly in an ionized form, the ionized form capable of being converted into an un-ionized form;
a carrier; and
a buffer system,
wherein the buffer system comprises at least two different buffering agents and is capable of changing the pH of saliva from an arbitrary initial pH to a predetermined final pH, independent of the arbitrary initial pH, and of sustaining the predetermined final pH for a period of time, and
wherein the buffer system favors substantially complete conversion of the ionized form to the un-ionized form.

2. The composition of claim 1 wherein the at least one therapeutic agent is basic.

3. The composition of claim 2 wherein the at least one therapeutic agent is selected from the group consisting of alfentanil, allylprodine, alphaprodine, anileridine, benzylmorphine, bezitramide, clonitazene, codeine, dextromoramide, diampromide, dihydrocodeine, dimenoxadol, dimepheptanol, dimethylthiambutene, dioxaphetyl butyrate, dipipanone, ethoheptazine, ethylmethylthiambutene, ethylmorphine, etonitazene, fentanyl, heroin, hydrocodone, isomethadone, levophenacylmorphan, lofentanil, meperidine, methadone, morphine, narceine, nicomorphine, norlevorphanol, normethadone, norpipanone, opium, oxycodone, papaveretum, phenadoxone, phenoperidine, piminodine, piritramide, propheptazine, promedol, properidine, propiram, propoxyphene, sufentanil, tramadol, tilidine, analogs, and mixtures thereof.

4. The composition of claim 3 wherein the at least one therapeutic agent is oxycodone.

5. The composition of claim 1 wherein the at least one therapeutic agent is acidic.

6. The composition of claim 5 wherein the at least one therapeutic agent is montelukast.

7. The composition of claim 1 wherein the at least one therapeutic agent is amphoteric.

8. The composition of claim 7 wherein the at least one therapeutic agent is selected from the group consisting of buprenorphine, butorphanol, cyclazocine, desomorphine, dezocine, dihydromorphine, eptazocine, hydromorphone, hydroxypethidine, ketobemidone, levallorphan, levorphanol, meptazinol, metazocine, metopon, morphine, nalbuphine, nalorphine, naloxone, naltrexone, normorphine, oxymorphone, pentazocine, phenomorphan, phenazocine, analogs, and mixtures thereof.

9. The composition of claim 2 wherein the predetermined final pH is within a range of from about 7.1 to about 11.5.

10. The composition of claim 2 wherein the predetermined final pH is within a range of from about 9 to about 11.

11. The composition of claim 5 wherein the predetermined final pH is within a range of from about 2 to about 6.9.

12. The composition of claim 5 wherein the predetermined final pH is within a range of from about 2 to about 4.

13. The composition of claim 1 wherein the carrier provides for a dosage form selected from the group consisting of a lozenge, a chewing gum, and a quick-dissolving tablet.

14. The composition of claim 13 wherein the carrier provides for a lozenge.

15. The composition of claim 13 wherein the carrier provides for a quick-dissolving tablet.

16. The composition of claim 13 wherein the carrier provides for a chewing gum.

17. The composition of claim 16 wherein the carrier is a gum base.

18. The composition of claim 17 wherein the gum base comprises at least one hydrophobic polymer and at least one hydrophilic polymer.

19. The composition of claim 18, wherein the at least one hydrophilic polymer and the at least one hydrophobic polymer are independently selected from the group consisting of a natural polymer, a synthetic polymer, and mixtures thereof.

20. The composition of claim 19, wherein the at least one hydrophobic polymer is selected from the group consisting of a butadiene-styrene copolymer, butyl rubber, polyethylene, polyisobutylene, polyvinyl acetate phthalate, and mixtures thereof.

21. The composition of claim 20 wherein the hydrophobic polymer comprises a mixture of butyl rubber and polyisobutylene.

22. The composition of claim 1 wherein the buffer system favors at least 80% conversion of the ionized form to the un-ionized form.

23. The composition of claim 22 wherein the 80% conversion occurs in 10 minutes or less.

24. The composition of claim 1 wherein the buffer system favors at least 95% conversion of the ionized form to the un-ionized form.

25. The composition of claim 24 wherein the 95% conversion occurs in 10 minutes or less.

26. The composition of claim 1 wherein the buffer system favors at least 99% conversion of the ionized form into the un-ionized form.

27. The composition of claim 26 wherein the 99% conversion occurs in 10 minutes or less.

28. The composition of claim 1 wherein the buffering agents are selected from the group consisting of a mixture of a weak acid and a salt of weak acid, and a mixture of a base and a salt of a weak base.

29. The composition of claim 28 wherein the buffering agents are independently selected from the group consisting of sodium carbonate, sodium bicarbonate, potassium carbonate, potassium bicarbonate, potassium citrate and mono basic potassium phosphate, magnesium oxide, magnesium carbonate, magnesium bicarbonate, alkaline starch, ascorbic acid, and mixtures thereof.

30. The composition of claim 29 where one buffering agent is sodium bicarbonate and one buffering agent is sodium carbonate.

31. The composition of claim 29 where one buffering agent is potassium bicarbonate and one buffering agent is potassium carbonate.

32. The composition of claim 29 wherein the buffering agents are in weight ratio of from about 2-1:1-2.

33. The composition of claim 29 wherein the buffering agents are in weight ratio of from about 3-1:1-3.

34. The composition of claim 29 wherein the buffering agents are in weight ratio of from about 5-1:1-5.

35. The composition of claim 29 wherein the buffering agents are in weight ratio of from about 10-1:1-10.

36. The composition of claim 29 wherein the buffering agents are in a 1:1 ratio by weight.

37. The composition of claim 1 wherein the period of time for sustaining the predetermined final pH is at least 5 minutes.

38. The composition of claim 1 wherein the period of time for sustaining the predetermined final pH is at least 10 minutes.

39. The composition of claim 1 wherein the period of time for sustaining the predetermined final pH is at least 20 minutes.

40. The composition of claim 1 further comprising a penetration enhancer.

41. A chewing gum composition comprising:
at least one therapeutic agent at least partly in an ionized form, the ionized form capable of being converted into an un-ionized form;
a gum base;
a protecting agent, wherein the protecting agent coats at least a portion of the therapeutic agent and reduces adhesion between the therapeutic agent and the gum base; and
a buffer system,
wherein the buffer system comprises at least two different buffering agents and is capable of changing the pH of saliva from an arbitrary initial pH to a predetermined final pH, independent of the arbitrary initial pH, and of sustaining the predetermined final pH for a period of time, and
wherein the buffer system favors substantially complete conversion of the ionized form to the un-ionized form.

42. The chewing gum composition of claim 41 wherein the protecting agent comprises magnesium stearate.

43. The chewing gum composition of claim 41 wherein the gum base is mixed into the therapeutic agent during its formulation so that the therapeutic agent is in an excess amount relative to the gum base.

44. The composition of claim 41 wherein the gum base comprises at least one hydrophobic polymer and at least one hydrophilic polymer.

45. The composition of claim 44, wherein the at least one hydrophilic polymer and the at least one hydrophobic polymer are independently selected from the group consisting of a natural polymer, a synthetic polymer, and mixtures thereof.

46. The composition of claim 45, wherein the at least one hydrophobic polymer is selected from the group consisting of a butadiene-styrene copolymer, butyl rubber, polyethylene, polyisobutylene, polyvinyl acetate phthalate, and mixtures thereof.

47. The composition of claim 46 wherein the hydrophobic polymer comprises a mixture of butyl rubber and polyisobutylene.

48. The chewing gum composition of claim 41 further comprising a sweetener.

49. The chewing gum composition of claim 48 wherein the sweetener is selected from the group consisting of a mono-polysaccharide, di-polysaccharide, tri-polysaccharide, non-saccharide-based sweetener, dipeptide, chlorinated sugar derivative, sugar alcohol, hydrogenated starch hydrolysate, 3,6-dihydro-6-methyl-1-1,2,3-oxathiazin-4-one-2,2-dioxide, and pharmaceutically acceptable salts, esters, analogs, and mixtures thereof.

50. The chewing gum composition of claim 41 further comprising a compound selected from the group consisting of a binder, a filler, a flavoring agent, a scenting agent, a coloring agent, a preservative, a plasticizer, a penetration enhancer, an elastomeric solvent, and mixtures thereof.

51. The chewing gum composition of claim 41 wherein the buffering agents are selected from the group consisting of a mixture of a weak acid and a salt of weak acid, and a mixture of a base and a salt of a weak base.

52. The chewing gum composition of claim 51 wherein the buffering agents are independently selected from the group consisting of sodium carbonate, sodium bicarbonate, potassium carbonate, potassium bicarbonate, potassium citrate and mono basic potassium phosphate, magnesium oxide, magnesium carbonate, magnesium bicarbonate, alkaline starch, ascorbic acid, and mixtures thereof.

53. The composition of claim 52 where one buffering agent is sodium bicarbonate and one buffering agent is sodium carbonate.

54. The composition of claim 52 where one buffering agent is potassium bicarbonate and one buffering agent is potassium carbonate.

55. The composition of claim 52 wherein the buffering agents are in weight ratio of from about 2-1:1-2.

56. The composition of claim 52 wherein the buffering agents are in weight ratio of from about 3-1:1-3.

57. The composition of claim 52 wherein the buffering agents are in weight ratio of from about 5-1:1-5.

58. The composition of claim 52 wherein the buffering agents are in weight ratio of from about 10-1:1-10.

59. The composition of claim 52 wherein the buffering agents are in a 1 to 1 ratio by weight.

60. The composition of claim 41 wherein the period of time for sustaining the predetermined final pH is at least 5 minutes.

61. The composition of claim 41 wherein the period of time for sustaining the predetermined final pH is at least 10 minutes.

62. The composition of claim 41 wherein the period of time for sustaining the predetermined final pH is at least 20 minutes.

63. The chewing gum composition of claim 41 wherein the at least one therapeutic agent is basic.

64. The chewing gum composition of claim 63 wherein the at least one therapeutic agent is selected from the group consisting of alfentanil, allylprodine, alphaprodine, anileridine, benzylmorphine, bezitramide, clonitazene, codeine, dextromoramide, diampromide, dihydrocodeine, dimenoxadol, dimepheptanol, dimethylthiambutene, dioxaphetyl butyrate, dipipanone, ethoheptazine, ethylmethylthiambutene, ethylmorphine, etonitazene, fentanyl, heroin, hydrocodone, isomethadone, levophenacylmorphan, lofentanil, meperidine, methadone, morphine, narceine, nicomorphine, norlevorphanol, normethadone, norpipanone, opium, oxycodone, papaveretum, phenadoxone, phenoperidine, piminodine, piritramide, propheptazine, promedol, properidine, propiram, propoxyphene, sufentanil, tramadol, tilidine, analogs, and mixtures thereof.

65. The chewing gum composition of claim 64 wherein the at least one therapeutic agent is oxycodone.

66. The chewing gum composition of claim 41 wherein the at least one therapeutic agent is acidic.

67. The composition of claim 66 wherein the at least one therapeutic agent is montelukast.

68. The composition of claim 41 wherein the at least one therapeutic agent is amphoteric.

69. The composition of claim 68 wherein the at least one therapeutic agent is selected from the group consisting of buprenorphine, butorphanol, cyclazocine, desomorphine, dezocine, dihydromorphine, eptazocine, hydromorphone, hydroxypethidine, ketobemidone, levallorphan, levorphanol, meptazinol, metazocine, metopon, morphine, nalbuphine, nalorphine, naloxone, naltrexone, normorphine, oxymorphone, pentazocine, phenomorphan, phenazocine, analogs, and mixtures thereof.
